(19) United States
(12) Patent Application Publication
(10) Pub. No.: US 2013/0317400 A1
(43) Pub. Date: Nov. 28, 2013

(54) Brace, Device, and Method for Correcting Poor Posture

(71) Applicant: Joseph Steven Ferezy, Urbandale, IA (US)

(72) Inventor: Joseph Steven Ferezy, Urbandale, IA (US)

(21) Appl. No.: 13/862,499

(22) Filed: Apr. 15, 2013

Related U.S. Application Data
(60) Provisional application No. 61/651,303, filed on May 24, 2012.

(51) Int. Cl. A61N 1/04

(52) U.S. Cl. CPC A61N 1/0484
USPC 602/2

(57) ABSTRACT

A device for correcting poor posture comprises a wearable garment comprising a back brace, a plurality of electrodes connected to the wearable garment and configured to electrically stimulate human muscles, and an electric muscle stimulation unit connected to the plurality of electrodes and configured to provide electrical power to the plurality of electrodes.

REFERENCE TO RELATED APPLICATION

This application claims the benefit of U.S. Provisional Patent Application No. 6/165,1303, filed May 24, 2012, entitled, “A posture brace offering corrective electric muscle stimulation (EMS) when sensing poor posture.”

### BACKGROUND OF THE INVENTION

The present invention relates to a brace worn to provide Electric Muscle Stimulation (EMS) for correcting poor posture.

Most people work or spend a great deal of time engaged in activities that promote poor posture, such as sitting at a desk, driving long distances, performing physical labor, etc. The most common postural defect is “sloughing” and “cervical poking” where one's shoulders roll forward and their head extends out in front of their body. Most people are aware of their poor posture and that poor posture leads to their neck and shoulder pain. However, most people cannot change or control their posture for more than a few minutes.

Most posture braces are strictly mechanical and try to correct posture by physically pulling back on the shoulders. This causes a “cutting in” effect and patient discomfort. They are uncomfortable and therefore compliance is poor.

As can be seen, there is a need for solutions to these and other problems.

### SUMMARY OF THE INVENTION

In one aspect of the present invention, a device for correcting poor posture comprises: a wearable garment comprising a back brace; a plurality of electrodes connected to the wearable garment and configured to electrically stimulate human muscles; and an electric muscle stimulation unit connected to the plurality of electrodes and configured to provide electrical power to the plurality of electrodes. In one aspect, the electric muscle stimulation unit is programmable via input controls. In one aspect, the device further comprises a plurality of contact sensors connected to the wearable garment and configured to detect movement or pressure suggesting poor posture of a user and to send a signal to activate selective electrodes attached to the electric muscle stimulation unit.

In one aspect, the plurality of contact sensors comprises a plurality of binary contact switches. In one aspect, the electric muscle stimulation unit is configured to provide electrical power to the plurality of electrodes at least in part due to the pressure signal from the plurality of contact sensors. In one aspect, the electric muscle stimulation unit is configured to derive a pressure profile against the back of the user and to provide a profile of electrical power to the plurality of electrodes according to both the pressure profile and an input signal input by the user.

In one aspect, a method of correcting poor posture comprises: providing the device as described; donning the wearable garment; and activating the electric muscle stimulation unit so as to provide electrical power to the plurality of electrodes. In one aspect, a method of correcting poor posture comprises: providing the device as described; donning the wearable garment; sitting in a posture; and activating the electric muscle stimulation unit so that the plurality of contact sensors sends a pressure signal to the electric muscle stimulation unit and the electric muscle stimulation unit provides electrical power to the plurality of electrodes to signal to the user to correct the posture. In one aspect, the method further comprises programming the electric muscle stimulation unit via input controls to provide electrical power to the plurality of electrodes according to a preference of the user.

These and other features, aspects and advantages of the present invention will become better understood with reference to the following drawings, description and claims.

### BRIEF DESCRIPTION OF THE DRAWINGS

FIG. 1: is a back view of one embodiment of the invention shown in use.

FIG. 2: is a perspective view of one embodiment of the invention showing the electrode 12.

FIG. 3: is a perspective view of one embodiment of the invention showing the contact switch 22.

FIG. 4: is a perspective view of one embodiment of the invention showing the EMS unit 14. FIG. 5: is a flowchart of one embodiment of the invention.

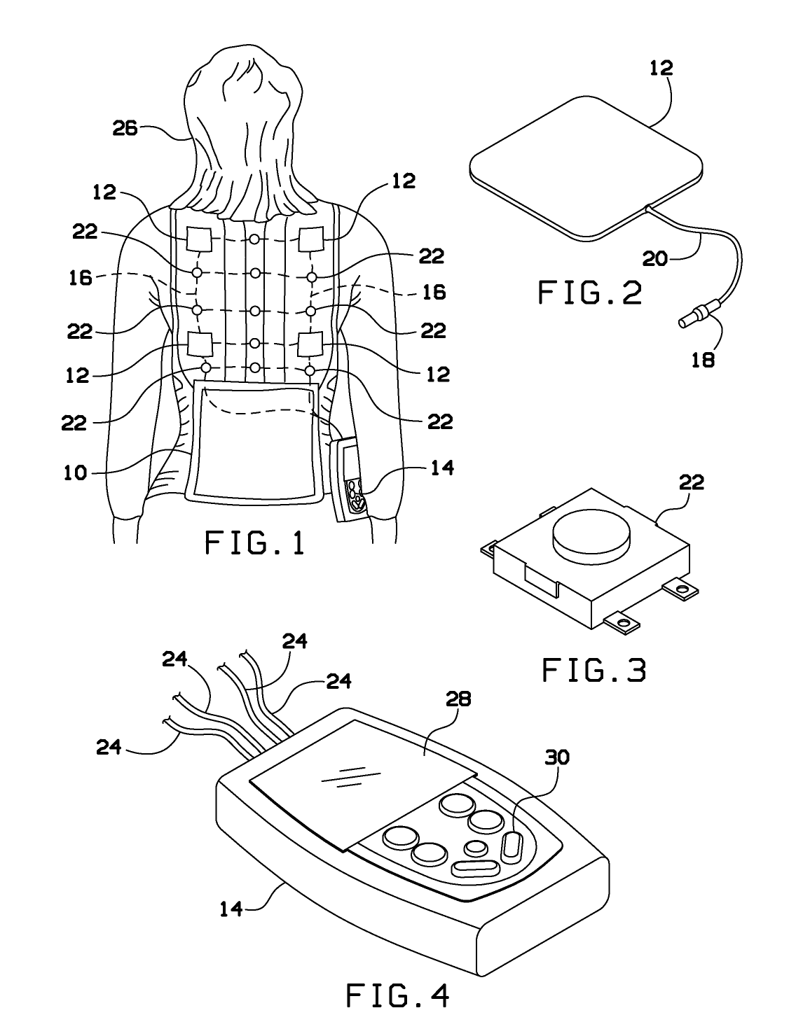

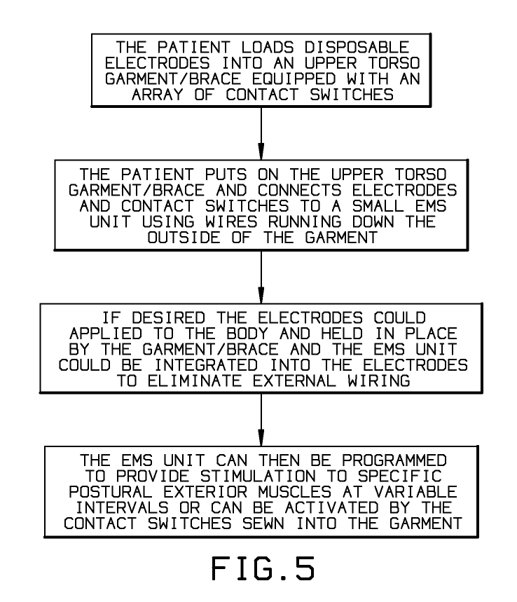

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention.

Referring now to the figures, the following reference numbers may refer to elements of the invention:

10: is the upper torso garment/brace.

12: are the electrodes.

14: is the EMS unit.

16: is the wiring inside the garment.

18: are the electrode connectors.

20: are the electrode lead wires.

22: are the contact sensors and/or switches.

24: is the EMS unit wiring.

26: is the person.

28: is the EMS unit screen.

30: are the EMS unit input controls.

A device according to one embodiment of the present invention includes a more comfortable electro-mechanical elastic brace that uses gentle support and the patient's own postural muscles through EMS to naturally extend the upper torso without patient discomfort, eventually permanently correcting poor posture.

A device according to one embodiment of the present invention is an elastic brace worn over the upper torso. Embedded in the brace are electrodes attached to an electrical muscle stimulator. The electrodes are spaced so that they cover the motor points of postural extensor muscles of the upper torso. When sensors in the advanced version of the device detect slouching, the EMS unit is activated and the appropriate muscles begin to contract to correct posture. The contractions last until posture is corrected (or longer, a feature which may be programmable). This not only strengthens weak postural extensor muscles but also reminds the patient to maintain consciousness of their posture, causing a “learned response” until eventually the brace does not have to be worn.

A device according to one embodiment of the present invention may include one or more of the following elements and/or features:
Supportive elastic upper torso garment/brace in variable sizes.
EMS electrodes and lead wires.
Battery operated EMS device.
Stretch sensitive contact or tactile switches.

Workings of the device according to one embodiment of the present invention may be described as follows. The patient loads disposable electrodes into the garment/brace and wears it like an undershirt. Elastic panels provide gentle postural support. Lead wires from the electrodes are run through holders on the outside of the garment to a small EMS unit which attaches to the garment/brace, a belt, or any other connection to the user's body and/or apparel. The EMS unit can be programmed to provide gentle muscle stimulation to eight specific postural extensor muscles at variable intervals, or can be activated by stretch sensor switches sewn into the garment/brace.

Making the device according to one embodiment of the present invention may be described as follows. Various sizes of elastic posture garment/braces may be manufactured with appropriate reinforcement, cut-outs and pockets sewn in. Also, contact switches activated by stretch may be sewn into the garment/brace as well. Existing EMS units and lead wires may be obtained and fitted to the appropriate sizes.

The device may be improved by adding some more solid plastic or metal support to the garment/brace. Also, a larger garment/brace that includes sensors for pelvic tilting and housing electrodes causing abdominal muscle contraction can be offered. The device can be used without any sensors or switches. In that case the EMS unit may be programmed to provide muscle stimulation as the patient desires.

Electrodes can be applied to the body and held in place by a brace. The EMS unit itself may be integrated into the electrode, thus eliminating the lead wire. The brace may be made of material other than elastic.

Use of the device according to one embodiment of the present invention may be described as follows. The person would simply load the electrodes, wear the garment/brace, plug in the wires, insert the batteries, and program and wear the EMS unit as they desire. Further, the invention may be designed for and used on animals other than humans.

In one aspect of the present invention, a device for correcting poor posture comprises: a wearable garment comprising a back brace 10; a plurality of electrodes 12 connected to the wearable garment and configured to electrically stimulate human muscles; and an electric muscle stimulation unit 14 connected to the plurality of electrodes 12 and configured to provide electrical power to the plurality of electrodes. The garment may comprise a vest, torso covering, or any other garment to which other elements (such as electrodes 12) may be attached; it may comprise any known material, such as fabric, synthetic materials, etc. The electrodes 12 may be configured to provide an electrical charge to the human muscles so as to cause the user to respond to the charge either voluntarily (low voltage) or involuntarily (high voltage). The EMS unit 14 may comprise a device that provides power to the electrodes 12 and may include a processor, memory storage, power source (e.g., batteries), transformers, input controls 30, a display or screen 28, and any other elements to allow the user to provide power to the electrodes 12 according to a preset configuration and/or program programmed by the user. In one aspect, the electric muscle stimulation unit is programmable via input controls 30.

In one aspect, the device further comprises a plurality of contact sensors 22 connected to the wearable garment and configured to detect a pressure against a back of a user and to send a pressure signal to the electric muscle stimulation unit 14. The contact sensors 22 may correspond to electrodes 12 or may be independent. They may provide information to the unit 14 about varying degrees or pressure, or may be binary contact switches that provide only on-off information to the unit 14. In one aspect, the electric muscle stimulation unit 14 is configured to provide electrical power to the plurality of electrodes 12 at least in part due to the pressure signal from the plurality of contact sensors 22. For example, if a contact sensor 22 located near the top right region of the user's back sends information to the unit 14 that the pressure is too high (or if the switch is closed in the case of a contact switch 22), then the unit 14 may respond by sending a charge to an electrode at or near the corresponding contact sensor 22 to correct the user's posture. In another more advanced aspect, the electric muscle stimulation unit 14 is configured to derive (via the processing power of the processor) a pressure profile against the back of the user based on the information coming from contact sensors 22, and then to provide a profile of electrical power to the plurality of electrodes 12 according to the pressure profile and/or an input signal input by the user. For example, the pressure profile might suggest that the user is slouching in a particular way that may be corrected by sending a particular electrical power profile to the electrodes 12. This electrical power profile may also depend on an input by the user (such as low voltage versus high voltage, waves of power to the electrodes 12, quasi-random power to the electrodes 12, etc.)

In one aspect, a method of correcting poor posture comprises: providing the device as described; donning the wearable garment; and activating (such as turning on) the electric muscle stimulation unit 14 so as to provide electrical power to the plurality of electrodes 12. In one aspect, a method of correcting poor posture comprises: providing the device as described; donning the wearable garment; sitting in a posture; and activating the electric muscle stimulation unit 14 so that the plurality of contact sensors 22 sends a pressure signal to the electric muscle stimulation unit 14 and the electric muscle stimulation unit 14 provides electrical power to the plurality of electrodes 12 to signal to the user to correct the posture. In one aspect, the method further comprises programming the electric muscle stimulation unit via input controls 30 to provide electrical power to the plurality of electrodes 12 according to a preference of the user.

It should be understood, of course, that the foregoing relates to exemplary embodiments of the invention and that modifications may be made without departing from the spirit and scope of the invention.

What is claimed is:

1. A device for correcting poor posture, comprising:
a wearable garment comprising a back brace;
a plurality of electrodes connected to the wearable garment and configured to electrically stimulate human muscles; and
an electric muscle stimulation unit connected to the plurality of electrodes and configured to provide electrical power to the plurality of electrodes.

2. The device as claimed in claim 1, wherein the electric muscle stimulation unit is programmable via input controls.

3. The device as claimed in claim 1, further comprising a plurality of contact sensors connected to the wearable garment and configured to detect a pressure against a back of a user and to send a pressure signal to the electric muscle stimulation unit.

4. The device as claimed in claim 3, wherein the plurality of contact sensors comprises a plurality of binary contact switches.

5. The device as claimed in claim 3, wherein the electric muscle stimulation unit is configured to provide electrical power to the plurality of electrodes at least in part due to the pressure signal from the plurality of contact sensors.

6. The device as claimed in claim 5, wherein the electric muscle stimulation unit is configured to derive a pressure profile against the back of the user and to provide a profile of electrical power to the plurality of electrodes according to both the pressure profile and an input signal input by the user.

7. A method of correcting poor posture, comprising:
providing the device as claimed in claim 1;
donning the wearable garment; and
activating the electric muscle stimulation unit so as to provide electrical power to the plurality of electrodes.

8. A method of correcting poor posture, comprising:
providing the device as claimed in claim 3;
donning the wearable garment;
sitting in a posture; and
activating the electric muscle stimulation unit so that the plurality of contact sensors sends a pressure signal to the electric muscle stimulation unit and the electric muscle stimulation unit provides electrical power to the plurality of electrodes to signal to the user to correct the posture.

9. The method as claimed in claim 8, further comprising programming the electric muscle stimulation unit via input controls to provide electrical power to the plurality of electrodes according to a preference of the user.
